(19) United States
(12) Patent Application Publication
(10) Pub. No.: US 2022/0023072 A1
(43) Pub. Date: Jan. 27, 2022

(54) ENVELOPING BODY WITH STIFFENING ELEMENTS

(71) Applicant: Ottobock SE & Co. KGAA, Duderstadt (DE)

(72) Inventors: Hendryk ENGELBART, Göttingen (DE); Lars Benjamin FINKE, Landolfshausen (DE)

(73) Assignee: Ottobock SE & Co. KGAA, Duderstadt (DE)

(21) Appl. No.: 17/296,958

(22) PCT Filed: Nov. 26, 2019
(86) PCT No.: PCT/EP2019/082596

§ 371 Date: May 25, 2021

(51) Int. Cl. A61F 2/78

(52) U.S. Cl. CPC A61F 2/7812; A61F 2002/5027

(57) ABSTRACT

The invention relates to an enveloping body for at least partially enveloping a limb, having an enclosed volume and a connection for supplying and removing fluid to the volume or from the volume, wherein the enveloping body forms an inner circumference and an outer circumference; on the enveloping body reinforcement elements are arranged that are designed to be brought into contact as a result of the removal of fluid from the volume or to increase their contact with one another as a result of the removal of liquid.

The invention relates to an enveloping body for at least partially enveloping a limb, having an enclosed volume and a connector for supplying and removing fluid to the volume and from the volume, the enveloping body forming an inner circumference and an outer circumference. The enveloping body is provided in particular to bear on a stump or on a limb.

Between a prosthesis socket and a limb stump, a liner is generally provided in order to cushion the stump, to compensate for irregularities in the socket surface and to produce a connection between the stump and the prosthesis socket, said liner being pulled on over the stump. The liner adheres to the skin surface of the stump and, at its distal end, can have locking elements, for example a pin or the like, that interacts with a corresponding mechanical locking device in the prosthesis socket in order to secure the liner and prevent the liner from moving out of the prosthesis socket counter to the direction of insertion. If the prosthesis socket is secured on the liner by a vacuum, a sealed-off volume is formed between the liner and the prosthesis socket, from which volume air is withdrawn via a pump or a valve. The vacuum has the effect that the prosthesis socket is held on the liner.

Prosthesis liners are generally made from an elastomer material, for example silicone, having a high degree of adherence to the stump. To put it on, the liner is rolled up, the distal and preferably closed end of the prosthesis liner is placed onto the stump end, and the liner is then unrolled over the stump. The liner generally has an internal diameter slightly smaller than the external diameter of the stump, in order to permit a relatively secure fit on the stump.

U.S. Pat. No. 5,387,245 A discloses a prosthesis liner comprising an inner liner and an outer liner made of an elastomer material. The inner liner and the outer liner are adhesively bonded to each other, wherein, in order to form a bladder between the inner liner and the outer liner, one region is not adhesively bonded. A valve is assigned to the bladder and allows air to be introduced into the bladder or released from the latter, so as to be able to compensate for fluctuations in the volume of the stump.

US 2013/0218296 A1 relates to a prosthesis with a socket and a liner, wherein a fluid is introduced in a region between the socket and the liner. The amount of fluid in the region can be regulated. To allow the prosthesis to be fitted more easily to the amputation stump, the inner circumference of the liner is widened, by means of fluid being pumped out of the region. Fluid is then conveyed back into the region in order to apply the liner firmly to the amputation stump.

EP 2 327 378 B1 relates to a cap for a prosthesis, which cap is applied to an amputation stump and secured to a prosthesis socket. The cap is composed of two membranes which form an airtight chamber. A fluid is introduced into the airtight chamber in order to improve the wearing comfort.

DE 917 687 B relates to a double-walled socket for a stump, with a hollow space which is divided into interconnected compartments by means of a series of woven fabrics. Arranged on the socket are a tube and a suction plug, via which the compartments of the hollow space can be inflated by means of a bellows or another pump.

US 2003/0078674 A1 relates to a prosthesis socket having, arranged on its inner face, a system of chambers into which a fluid is introduced. When the prosthesis socket is applied to an amputation stump, the amounts of fluid in the chambers can be altered. A change in the stump volume can thus be compensated, in order to ensure that the prosthesis is comfortable to wear.

U.S. Pat. No. 1,057,562 A describes a stump support composed of a rectangular inflatable cushion having a length greater than the circumference of the leg, such that the inflatable cushion can be placed with overlapping around the stump. The cushion extends over and rests upon an upper edge of a prosthesis socket and is furnished with a valve in the region of the upper edge.

U.S. Pat. No. 5,108,456 A relates to a prosthesis having a hard outer shell and a socket arranged in the latter, wherein a large number of inflatable air chambers are arranged on the socket. The air chambers can be inflated and emptied in order to improve the wearing comfort and the stability of the prosthesis applied to an amputation stump.

SU 731 963 relates to a prosthesis for a lower limb. Inflatable hoses are arranged in a zigzag arrangement in a rigid prosthesis socket in order to facilitate adaptation to the stump.

In the prior art there are also bandages or cuffs that are placed around a limb in order to exert a circumferential pressure on the limb. Such bandages or cuffs are preferably produced from a woven fabric or a foamed material. To fit them in place, these enveloping bodies, which have both a proximal opening and a distal opening, are likewise stretched.

DE 10 2016 101 670 A1 relates to an enveloping body for at least partially enveloping a limb, said enveloping body having an enclosed volume and a connector for supplying fluid into the volume and removing fluid out of the volume. The enveloping body forms an inner circumference and an outer circumference, wherein the inner circumference of the enveloping body increases as the pressure of the volume increases, so as to make the enveloping body easier to apply.

US 2016/0270479 A1 relates to a shoe that is intended to be easy to put on and easy to take off. It is to be inexpensive, light and comfortable to wear and is intended to promote health and to be adjustable in length. This is achieved by the fact that the shapes of an outer layer and of inner volumes are arranged corresponding to various body parts. The outer layer and also the volumes are made of a stretchable material and are joined mainly to the inside of the outer layer. Air can be introduced into or withdrawn from the volumes via valves. When air is introduced into the volumes, the whole shoe expands on account of the flexibility, making it easier to put the shoe on. After the foot has been inserted, the valves are opened and air is forced out by means of the volumes contracting. The volumes, together with the outer layer of the shoe, come into close contact with the foot, such that the shoe can be worn without slipping.

An advantage of the solutions from the prior art lies in the high level of flexibility and elasticity of the enveloping bodies such as cuffs or liners. A disadvantage is the need for a rigid prosthesis socket or frame in order to be able to secure further components to a limb.

The object of the present invention is therefore to make available an enveloping body which, on the one hand, permits individual adaptation to the respective stump and, on the other hand, facilitates simple coupling of further components.

According to the invention, this object is achieved by an enveloping body having the features of the main claim. Advantageous embodiments and developments of the invention are disclosed in the subclaims, the description and the figures.

In the enveloping body according to the invention for at least partially enveloping a limb, having an enclosed volume and a connector for supplying and removing fluid to the volume and from the volume, the enveloping body forming an inner circumference and an outer circumference, provision is made that, on the enveloping body, stiffening elements are arranged which are designed to be brought into contact as a result of removal of fluid from the volume or to strengthen their contact with one another as a result of removal of fluid. By bringing the stiffening elements into contact with one another or strengthening the contact of the stiffening elements with one another after fluid has been drained off or evacuated, e.g. by pumping, from the volume, it is possible to adapt the liner to the current shape of the stump upon each fitting and when so required, and at the same time to make available a stiffened, optionally rigid form of the enveloping body, such that in the applied state, after the stiffening elements have been brought into contact or after the contact or the pressing force has been increased, sufficient stiffness is present on the enveloping body to ensure that further components, such as orthotic or prosthetic joints, can be applied to the enveloping body and can be secured thereon. With the enveloping body, it is possible to combine the variable behavior of an elastic liner with the rigid behavior of a prosthesis socket such that, with an enveloping body of standard design, an individual adaptation to the respective user can be achieved in a state when the enveloping body is not evacuated or free of pressure or when the volume is provided with an overpressure. In a filled or pressure-free state of the volume, the enveloping body can be widened or adapted flexibly to the limb. When the fluid is drained off, the enveloping body bears on the limb and the stiffening elements are brought into contact with one another or, if they are already in contact, the pressing forces are increased. If a vacuum is actively generated in the volume relative to the environment, increased pressing and stiffening of the enveloping body is obtained. After the evacuation, a more stable, optionally stiffened or rigid structure forms, such that the enveloping body can serve as a prosthesis socket or as a dimensionally stable support for the enclosed limb. After adaptation to the volume of the enclosed limb or of the stump, the liner can be formed reversibly to a rigid envelop or a socket.

In one embodiment of the invention, the stiffening elements are arranged inside the volume, as a result of which a smooth inner and outer contour can be achieved while at the same time protecting the stiffening elements from external influences. Alternatively or in addition, the stiffening elements are arranged on the outer circumference of the enveloping body and, through the removal of fluid from the volume, are brought into contact with one another, or their contact is strengthened, when the outer circumference decreases on account of the fluid being drained off.

The enveloping body can have at least two walls between which the volume is enclosed, wherein the stiffening elements are arranged for example on the side directed toward the volume and/or are integrated in said side. It is thereby possible that the respective outer sides of the enveloping body can be formed without being influenced by the configuration of the stiffening elements; the inner side of the enveloping body is preferably designed with a smooth wall in order to permit uniform application to the limb or the stump. Fastening elements or receptacles for further components can be arranged on the outer side of the enveloping body in order to connect the enveloping body to further components. The stiffening elements are preferably arranged on mutually opposite and facing sides of the two walls or as a separate layer between the walls and, by removal of fluid or evacuation of the volume, are brought into contact with one another or with the opposite wall, in order to stiffen the enveloping body. A compact design can be obtained if the stiffening elements are integrated in the walls or in at least one wall.

In a development of the invention, provision is made that stiffening elements lying opposite one another or arranged next to one another in the volume can be brought into form-fit engagement with one another. The stiffening elements do not need to be arranged on mutually opposite inner walls of the volume; it is also possible that the stiffening elements are arranged in a scaled formation over one another or next to one another and thus, in the non-evacuated state of the enveloping body, are secured movably relative to one another on only one wall of the enveloping body. Only when the stiffening elements are brought into contact with one another, either on mutually opposite sides or by one wall of the volume being pressed onto the stiffening elements located inside the volume, or only when stiffening elements arranged next to one another are placed onto one another or engaged with one another by circumferential reduction, the mobility of the stiffening elements relative to one another is impeded or prevented and a stiffening of the whole enveloping body is achieved at least in the region of the stiffening elements. The stiffening elements lock onto one another and thereby prevent a movement relative to one another. The stiffening elements are secured, for example, on a wall, for example by cohesive bonding or by form-fit engagement, and transmit forces and moments from the limb to the outer circumference and/or vice versa. The stiffening elements are preferably rigid, dimensionally stable components or elements which transmit forces and moments without deforming or without permitting any appreciable deformation, in order thereby to cause a stiffening of the enveloping body in combination with other stiffening elements which are brought into contact with one another or whose pressing forces are increased. The stiffening elements can also be connected by other mechanisms to the walls in order to transmit forces or moments, for example by friction or by being pressed on by vacuum.

In one variant, the connector is designed as a suction connector with a closure device which, after the volume is evacuated and the stiffening elements are pressed onto one another, prevents a return flow of fluid into the volume. Opening of the connector may in some cases not be enough to generate sufficient pressing together of the stiffening elements or to bring the stiffening elements into sufficient contact with one another. Although the draining off of the fluid causes a reduction in the volume and therefore also in the outer circumference of the enveloping body, it is also possible that the resulting strengthening, stabilizing and stiffening is not enough for the desired purpose of use. Therefore, in one variant, the connector is designed as a suction connector which can be fluidically connected to a suction device, for example a vacuum pump, in order to suck further fluid out of the volume and to produce an internal pressure, inside the volume, that is below the ambient pressure. The greater the pressure difference between the volume and the environment, the more firmly the stiffening elements are brought into contact with one another or the more their contact with one another is strengthened, such that the stiffening effect increases. The shape adopted by the enveloping body on the limb is fixed by the evacuation, and stiffening is brought about.

On their contact faces, the stiffening elements can have mutually corresponding surface structures, for example knobs, webs, blocks, projections and undercuts, hook structures, parallel bristles or struts, embossed fabric, structured nonwovens, weaves or knits with a common nap orientation. When these surface structures are placed onto one another, the enveloping body stiffens at least in some regions. Alternatively or in addition, the stiffening elements have, at least on their contact faces, a coefficient of friction that is greater than or equal to 0.3. The coefficient of friction is chosen to be as high as possible, even though the adhering and holding conditions are additionally influenced by the geometry of the stiffening elements, the surfaces and the contact pressure. For the stiffening elements lying on one another or to be brought into contact with one another, a material pairing is preferably chosen that makes a movement of the stiffening elements relative to one another as difficult as possible. The stiffening elements can be formed from different materials or can be provided with regions having different materials, such that a material pairing at the contact faces is made available having a coefficient of friction that is as high as possible, in order to avoid, or at least to make difficult, a shifting of the stiffening elements relative to one another upon contact.

The stiffening elements not located in contact with one another are preferably arranged movably relative to one another on or in the enveloping body. If the stiffening element is formed of several components that are secured to the same wall, these components are brought into contact with one another and stiffen the enveloping body. The same applies to similarly designed stiffening elements on mutually opposite walls, such that the modular stiffening elements are brought into contact at both ends, upon evacuation of the volume, by being placed on one another or locked onto one another. A combination of stiffening effects can be obtained involving components of one stiffening element being placed on one another or brought into contact and mutually opposite stiffening elements being placed on one another and brought into contact.

In a development of the invention, provision is made that the enveloping body has an inner wall and an outer wall which enclose the at least one volume. The at least one volume can have interconnected regions and/or can extend helically in the proximal-distal direction. For this purpose, the at least one volume can be divided by at least one intermediate wall into different but interconnected regions which, for example, are connected to one another by flow channels or valves. By means of the design with valves, it is possible to set different pressure levels within the volume. The regions preferably extend over the entire length of the enveloping body, i.e. from a proximal end to a distal end, wherein the volume or the regions can be configured in the manner of a coiled spring or a thread.

In addition, there is the possibility that the enveloping body has a plurality of volumes which are distributed about the circumference and are separated by at least one intermediate wall, such that individual chambers are formed which are distributed about the circumference, making it possible to carry out a different evacuation or to have individual chambers not evacuated at all.

Alternatively or in addition, chambers can be arranged radially one after another, for example in a ring shape or a partial ring shape around the circumference of the stump or of the limb, particularly if the enveloping body is designed as a closed cross section with a tube-like contour. Then, two chambers are formed by three circumferentially continuous walls, for example. These annular walls form annular gaps between one another, which annular gaps form two volumes that can be filled with fluid or can be evacuated. By means of further annular walls, it is possible to add further volumes. The individual annular gaps or annular volumes can be separated from one another by intermediate walls or partitions, such that a large number of chambers distributed about the circumference of the enveloping body can be formed in a different thickness and different width.

The inner wall and/or intermediate wall and/or outer wall are/is foldable and/or elastic, in order to permit simple deformation, widening and evacuation. In a development of the invention, in order to obtain an enveloping body that can be adapted as individually as possible, each volume is assigned a connector for the supply and removal of fluid. The individual volumes can be connected to one another via valves, for example adjustable valves, and can permit flows of fluid through individual chambers into other chambers.

In a development of the invention, provision is made that the stiffening elements are designed as struts, plates, granules, filaments, wovens, knits and/or structured surface material.

The inner circumference of the enveloping body can increase as the pressure of the volume increases, in order to facilitate the application of an enveloping body, particularly if the inner circumference is designed as a closed inner circumference and the enveloping body is designed as a liner or cuff. Application is thus made easier, since the inner circumference is increased and the limb or the stump can be easily inserted into an enlarged inner hollow space. After fluid has been drained off from the volume or from the volumes, the inner circumference of the enveloping body bears on the stump or the limb and, by virtue of the flexible and preferably elastic design of the inner wall, conforms to the particular contour.

The enveloping body can have an at least partially adhesive inner surface such that, after the enveloping body has been fitted in place, a relative movement with respect to the skin surface is avoided or reduced.

The enveloping body can be designed as a prosthesis liner, bandage, cuff, item of clothing or shoe. In an embodiment of the enveloping body as a prosthesis liner, a fastening device for a further prosthesis component that is to be secured is preferably arranged or formed on the enveloping body

In order to facilitate an enlargement of the inner hollow space of an enveloping body upon enlargement of the enclosed volumes, elements transmitting tensile force are arranged in or on the enveloping body, which elements can be rigid under tension or elastic. The elements transmitting tensile force are preferably arranged between two walls, e.g. between an inner wall and an outer wall or between an intermediate wall and an inner and/or outer wall, of the enveloping body and are located inside a volume enclosed by the walls. Thus, upon expansion of the enveloping body on account of an increase in pressure, the volume for the limb that is to be received or for the stump that is to be received increases.

Illustrative embodiments of the invention are explained in more detail below with reference to the attached figures, in which:

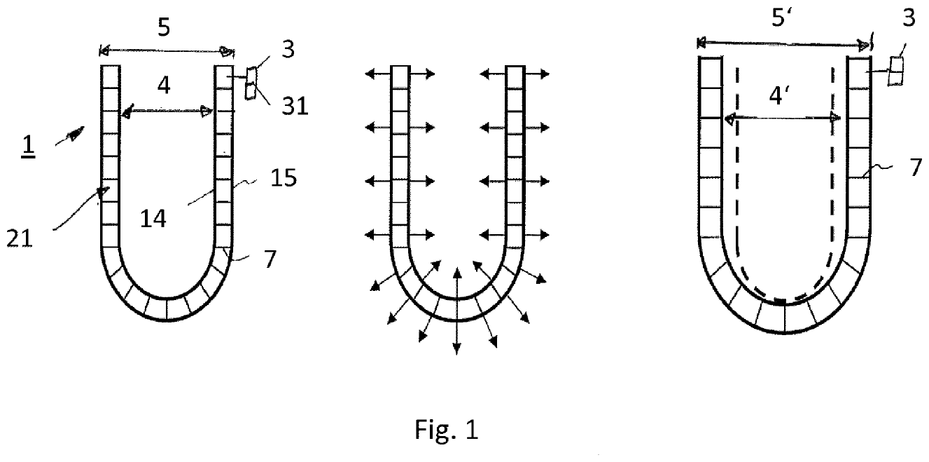

FIG. 1 shows a schematic view of an enveloping body in the form of a prosthesis liner, in two states;

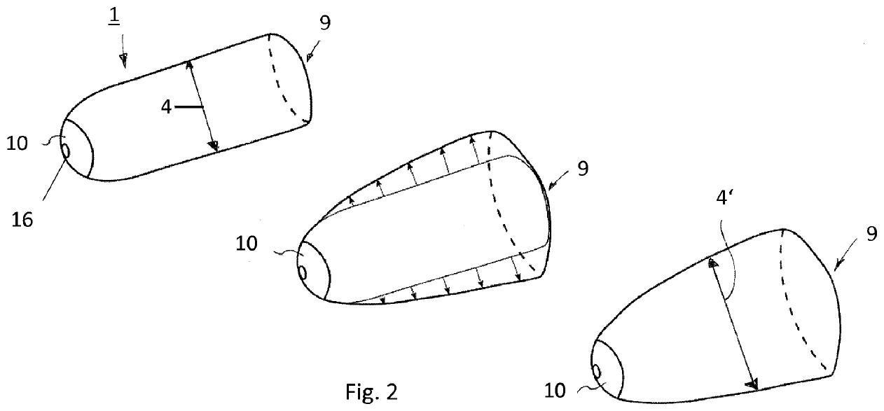

FIG. 2 shows a perspective view of an enveloping body as a prosthesis liner;

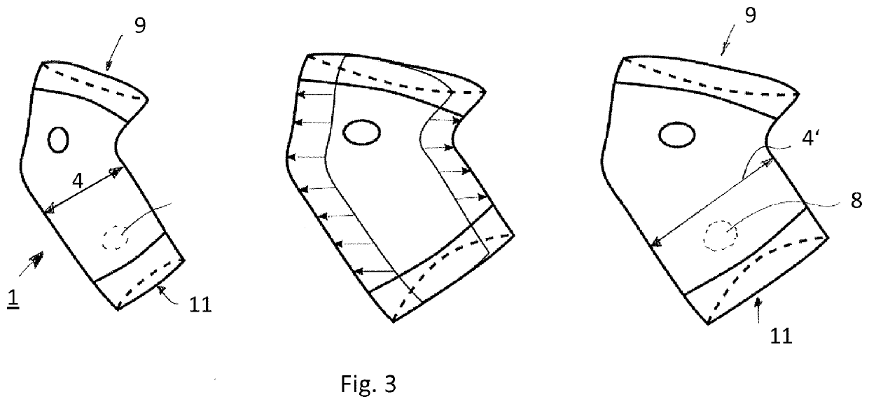

FIG. 3 shows a schematic view of a bandage;

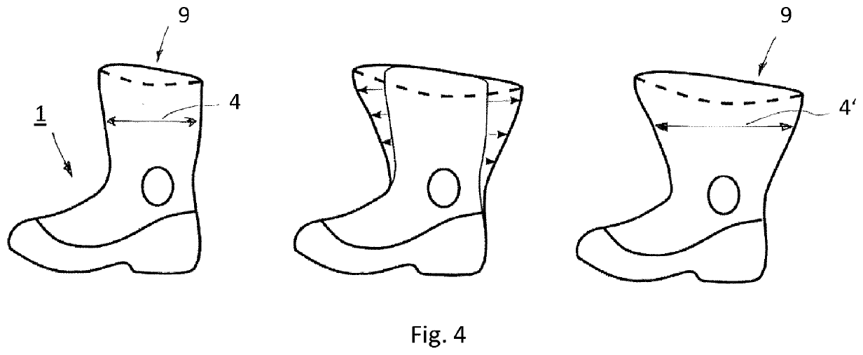

FIG. 4 shows a schematic view of an enveloping body in the form of a shoe;

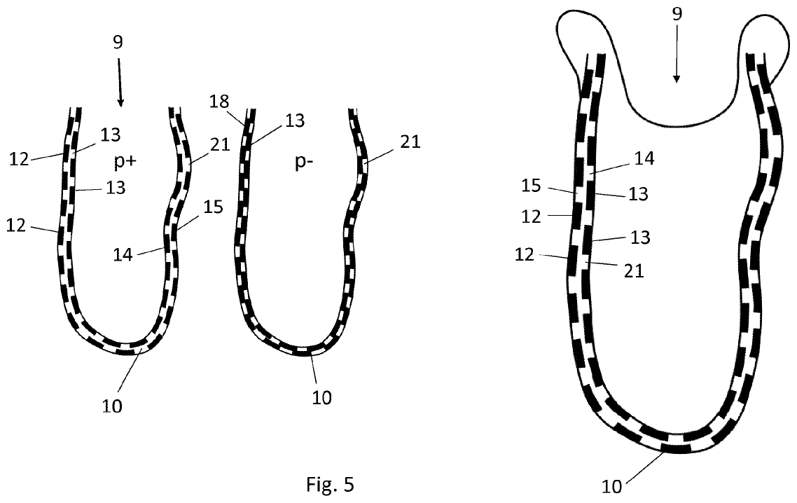

FIG. 5 shows a sectional view through a liner with stiffening elements;

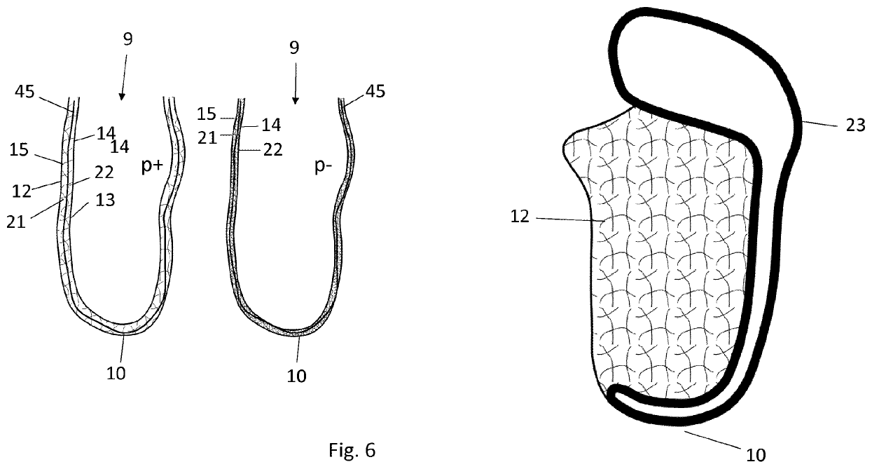

FIG. 6 shows a variant of the liner with an intermediate wall; and

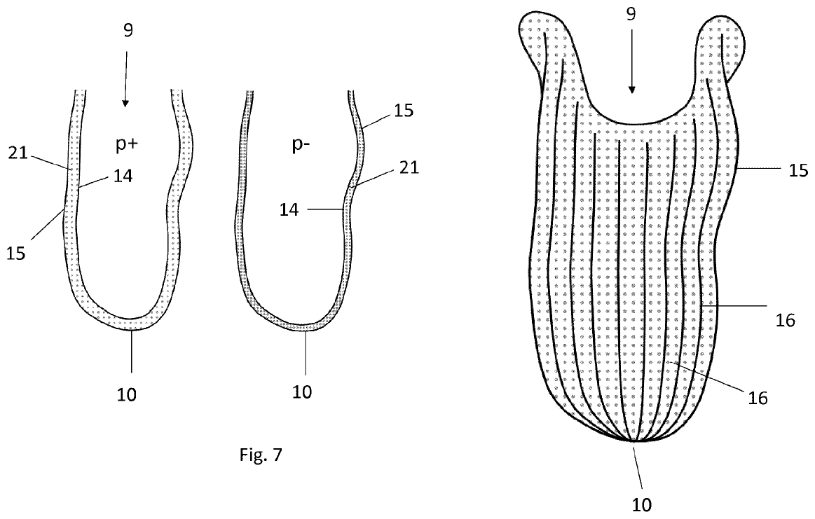

FIG. 7 shows a variant of the invention with a reinforcement.

FIG. 1 shows a schematic view of an orthopedic enveloping body 1 in the form of a prosthesis liner as a basic implementation of the invention. The enveloping body 1 has an inner wall 14 and an outer wall 15, which between them enclose a volume 21. The enveloping body 1 is thus double-walled and has a connector 3 with a closure device 31 in the form of a valve in order to fill the volume 21 between the inner wall 14 and the outer wall 15 with a fluid or to drain fluid from the volume 21. The fluid is preferably ambient air. A large number of elements 7 for transmitting tensile force are formed between the inner wall 14 and the outer wall 15 and, in the illustrative embodiment shown, are configured as webs. Instead of the webs, there is also the possibility of providing straps, pins or also just connection points between the inner wall 14 and the outer wall 15. In the state when not filled with fluid, the inner wall 14 can bear on the outer wall 15.

The enveloping body 1 is U-shaped in longitudinal section, while it has a substantially circular, closed contour in the cross section perpendicular to the longitudinal extent of the enveloping body 1. In the unfilled state shown, an inner circumference 4 and an outer circumference 5 are thus present; the dimensions of the circumferences 4, 5 derive from the shape, size and material properties.

When fluid is introduced into the closed-off volume 21 via the connector 3, the pressure inside the volume 21 increases since both the inner wall 14 and the outer wall are made of an elastic material, preferably an elastomer, or a combination of elastic portions and non-elastic portions. As a result of the increased internal pressure, corresponding forces act on the inner wall 14 and the outer wall 15; greater forces act on the outer wall 15 on account of the latter having a larger surface area. With identical material properties in terms of elongation, for example when the inner wall 14 and the outer wall 15 are made of the same material, greater deformation and elongation take place on account of the greater forces acting on the outer wall 15, which has the effect that the enveloping body 1 as a whole is stretched out. This is shown on the right in the figure. In the state when subjected to pressure, the outer circumference 5′ is increased compared to the view on the left. The inner circumference 4′ is also increased by comparison with the initial state, which is indicated by the broken line, as also is the distance between the inner wall 14 and the outer wall 15.

Through the pressure applied via the valve 31 and the connector 3, it is possible to increase the internal diameter and thus also the circumference 4 of the receiving space of the enveloping body 1 for the limb or the stump, such that the stump (not shown) can be easily inserted. When the valve 31 at the access 3 is opened, air escapes from the volume 21 on account of the elastic restoring forces that are made available by the inner wall 14 and the outer wall 15. The volume of the receiving space for the stump decreases, the inner wall 14 bears on the stump (not shown), and a secure fit of the enveloping body 1 on the stump is permitted. Depending on the degree of pressure reduction, a suitable pressing force of the inner wall 14 on the stump is obtained, wherein the maximum pressing force is obtained when the volume 21 is minimized.

FIG. 2 shows an embodiment of the enveloping body 1 in the form of a prosthesis liner with a proximal insertion opening 9 and a closed distal end region 10. A dimensionally stable cap and a fastening device 16 such as a pyramid adapter for mechanical locking to other prosthesis components such as knee joints or the like can be arranged at the distal end region 10. In the view at the top, the prosthesis liner 1 has a first circumference 4. From the distal end region 10, a side wall extends substantially conically in the direction of the proximal access opening 9. The side wall or the side wall region has a double-walled configuration; the distal end region 10 is either formed as one layer, or the two layers of a double-walled prosthesis liner are bonded adhesively to each other or joined cohesively to each other. No pressure fluid can be introduced in the distal end region 10.

The middle view in FIG. 2 shows the acting forces when a pressure fluid is introduced into the hollow space formed in the side wall or into the volume enclosed between an inner liner and an outer liner. The view on the right shows the shape of the prosthesis liner 1 at an increased internal pressure in the volume. The inner circumference 4′ is greater than the initial inner circumference 4; the access opening 9 of the prosthesis liner closed in the distal end region 10 is considerably larger than the original access opening 9. This makes placing the stump into the prosthesis liner 1 easier. After the stump end has made contact with the inner face of the distal end region 10, the valve 31 at the connector 3 is opened, the air escapes, and the side wall of the prosthesis liner 1 conforms to the stump.

An alternative embodiment of the invention is shown in FIG. 3, in which the enveloping body 1 is configured in the form of a pneumatically modifiable bandage. The bandage can be configured as a knee bandage and bridge a joint. It can alternatively be configured as an elbow bandage, a wrist bandage or an ankle bandage. An additional possibility is that the bandage is not configured to bridge a joint and instead can engage completely around the upper leg, upper arm, lower leg or forearm, for example. The cross section of the bandage is closed in the illustrative embodiment shown. The bandage 1 is double-walled along its entire length, between the proximal access opening 9 and the distal exit opening 11, and can have pressurized air applied to it via a connector (not shown). The application of pressurized air is shown in the middle view in FIG. 3, which illustrates how the bandage 1, from the initial state shown in the left-hand view, is converted to the end state shown in the right-hand view. The inner circumference 4′ in the pressurized state according to the view on the right is greater than the initial circumference 4, such that the bandage can be easily applied. Compression takes place by release of pressurized air. Regions 8 of increased adhesiveness can be formed on the inner face of the bandage 1, such that the enveloping body 1 has an adherent coating at least partially on its inner surface 8.

FIG. 4 shows a further variant of the invention, in which the enveloping body 1 is configured as a pneumatically operated shoe or boot. The shank region of the enveloping body 1, extending above the natural ankle joint, has a double-walled configuration and forms a sleeve-like volume that can be filled with pressurized air. The filling process is shown in the middle view, while the view on the right shows how the proximal access opening 9 is considerably increased in size compared to the initial extent. The inner circumference 4′ is likewise increased in relation to the original inner circumference 4, such that insertion into the shoe or boot 1 is made much easier. After insertion, the pressure in the volume 21 can be reduced, as a result of which the shank conforms to the lower leg.

FIG. 5 shows, on the left, a partial sectional view of an enveloping body 1 in the form of a prosthesis liner with a filled volume 21, as is indicated by the sign p+, while the middle view in FIG. 5 shows the section in an evacuated state in which the volume 21 has been freed of the fluid located therein. This is illustrated by the sign p−. Inside the volume 21 formed between the inner wall 14 and the outer wall 15, stiffening elements 12 are arranged which lie opposite one another inside the volume 21. In the partial sectional view on the left, the stiffening elements 12, 13 are spaced apart from one another since a fluid, for example air, is located inside the volume 21 and for example forms an overpressure in relation to the environment. In the illustrative embodiment shown, the stiffening elements 12, 13 are designed as separate components, for example as individual plates on the inner side of the outer wall 15 and on the side of the inner wall 14 directed toward the volume. If the fluid inside the volume 21 is now drained off, for example through the connector 3 (not shown) to which a pump can be attached, the inner wall 14 adopts the individual shape of the enclosed stump. Thereafter, or at the same time, the two walls 14, 15 bear on each other and bring the stiffening elements 12, 13 into contact with one another or strengthen the contact. If a vacuum is then applied, the inner wall 14 and the outer wall 15, with the stiffening elements 11, 12 located thereon or therein, are pressed onto one another. When the stiffening elements 12, 13 which are arranged on mutually opposite sides of the respective wall 14, 15 are pressed together or brought into increased contact, the enveloping body 1 stiffens and forms a dimensionally stable envelop for the stump. Alternatively to a pump, the fluid from the volume 21 can also be forced out of the volume 21 as a result of elastic restoring forces, for example by clamping devices which are arranged between the inner wall 14 and the outer wall 15 and act against a movement of the inner wall 14 away from the outer wall 15. After the evacuation of the volume 21 or of the volumes, the valve or the closure device 31 prevents a return flow of fluid, in particular ambient air, such that the rigid form of the enveloping body is maintained until the valve 31 is opened and the stiffening elements 12, 13 are able to move relative to one another.

The stiffening elements 12, 13 on the respective wall can be designed either as mutually movable individual plates, knobs, imbricated plates or other components which, in the emptied state of the volume 21, come into contact with one another or engage with one another and impede or prevent shifting of the individual components relative to one another. The stiffening elements 12, 13 can also be designed in one piece, and it is likewise possible that the stiffening elements 12 are arranged only on one wall if the stability of the stiffening elements 12, 13 brought into contact with one another is sufficient. The stiffening elements 12, 13 can also bear on one another in the filled state of the volume 21, but they are then preferably movable relative to one another and, by evacuation and by the respective other wall bearing on the stiffening elements 12, 13, are pressed onto one another and brought into contact, such that shifting of the individual components of the stiffening elements 12, 13 relative to one another is prevented or impeded.

In the view on the right in FIG. 5, a complete prosthesis liner is shown in partial cross section with the stiffening elements 12, 13, in the filled state of the volume 21. The stiffening elements 12, 13 can also be designed as auxetic surfaces.

FIG. 6 shows a variant of the invention in which an intermediate wall 45 is arranged between the outer wall 15 and the inner wall 14, such that two volumes 21, 22 form in the enveloping body 1. The closed volumes 21, 22 can be filled or emptied separately through a separate connector 3. Stiffening elements 12, 13 in the form of felt layers or fiber layers of directed orientation are arranged inside the volumes 21, 22 and, in the filled state of the volumes 21, 22 as shown in the view on the far left, allow the walls 14, 15, 45 to move relative to one another and, on account of the mobility of the walls 14, 15, 45, permit simple engagement and application of the enveloping body 1 in the form of a prosthesis liner.

If the fluid is then sucked out of the volumes 21, 22 as is shown in the middle view, the surfaces of the walls 14, 15, 45 provided with textile elements bear on one another and lock onto one another, such that the stiffness of the whole enveloping body is increased. Alternatively to an arrangement of textile elements such as felt, structure textiles or the like, it is possible to introduce knob-like films, ribbed surfaces or other surface structures that produce a locking action into the volumes 21, 22 and, by evacuation or by releasing the internal pressure, to move them toward one another and increase the stiffness by placing them onto one another. A fastening device 16 such as a pyramid adapter can be arranged at the distal end 10. However, the fastening device 16 can in principle also be arranged at other locations, in particularly laterally, of the enveloping body. The view on the right in FIG. 6 shows an illustrative embodiment of an enveloping body as a frame socket with a frame 23 and a dorsal socket with a distal end region 10 for receiving a stump end. The fixed frame does not completely enclose the limb here; the remaining part is formed only by the enveloping body equipped with stiffening elements 12 and is thus flexible in the state when filled with fluid.

FIG. 7 shows a further variant of the invention, in which the views on the left and in the middle each show different internal pressures present in the volume 21. Arranged inside the volume 21 are granules which, upon evacuation of the volume 21 as shown in the middle figure, are compressed, as a result of which a movement of the granule particles relative to one another is prevented. The stiffness thus increases, since the stiffening elements in the form of the granule particles are brought into contact with one another or are pressed onto one another. Filament-like reinforcements 16 can be arranged or embedded in the outer wall 15 or on the outside of the outer wall 15, in order to provide length stability of the enveloping body 1. Lengthening of the enveloping body 1 is then no longer possible, or is possible only to a limited extent. By contrast, radial widening when the pressure inside the volume 21 increases is possible.

The filament reinforcement 16 can also be designed to be radially continuous and produce connecting walls or partition walls between the inner wall 14 and the outer wall 15, such that a number of individual chambers separated from one another are distributed about the circumference, with the result that a large number of volumes are formed inside the enveloping body 1. Each volume can be provided with an outlet or a valve in order to fill or empty it separately or to provide it with an underpressure when a vacuum pump is applied.

All the illustrative embodiments of FIGS. 5-7 can also be used in the examples of enveloping bodies in FIGS. 1-4, particularly in respect of the stiffening elements.

1. An enveloping body for at least partially enveloping a limb, the enveloping body having an enclosed volume and a connector for supplying and removing fluid to the volume and from the volume, the enveloping body forming an inner circumference and an outer circumference,
wherein on the enveloping body, stiffening elements are arranged which are designed to be brought into contact as a result of removal of fluid from the volume or to strengthen their contact with one another as a result of removal of fluid.

2. The enveloping body as claimed in claim 1, wherein the stiffening elements are arranged inside the volume and/or on the outer circumference thereof.

3. The enveloping body as claimed in claim 1 wherein the enveloping body has at least two walls between which the volume is enclosed, and the stiffening elements are arranged on a side directed toward the volume and/or are integrated in said side.

4. The enveloping body as claimed in claim 1 wherein stiffening elements lie opposite one another in the volume or stiffening elements arranged next to one another can be brought into form-fit engagement with one another.

5. The enveloping body as claimed in claim 1 wherein the connector is designed with a closure device which, after the volume is evacuated and the stiffening elements are pressed onto one another, preventing a return flow of fluid into the volume.

6. The enveloping body as claimed in claim 1 wherein on their contact faces, the stiffening elements have mutually corresponding surface structures and/or a coefficient of friction μ≥0.3.

7. The enveloping body as claimed in claim 1 wherein stiffening elements that are not located in contact with one another are arranged to be movable relative to one another on or in the enveloping body.

8. The enveloping body as claimed in claim 1 wherein the enveloping body has an inner wall and an outer wall which enclose the volume.

9. The enveloping body as claimed in claim 1 wherein the volume has interconnected regions and/or extends helically in the proximal-distal direction.

10. The enveloping body as claimed in claim 1 wherein the enveloping body has a plurality of volumes which are distributed about the circumference and/or arranged radially one after another and are separated by at least one intermediate wall.

11. The enveloping body as claimed in claim 10, wherein the inner wall and/or intermediate wall and/or outer wall are/is foldable and/or elastic.

12. The enveloping body as claimed in claim 10, wherein each volume has a connector for the supply and removal of fluid.

13. The enveloping body as claimed in claim 1 wherein the stiffening elements are designed as struts, plates, granules, filaments, wovens, knits and/or structured surface material.

14. The enveloping body as claimed in claim 1 wherein the inner circumference of the enveloping body increases as a pressure of the volume increases.

15. The enveloping body as claimed in claim 1 wherein the enveloping body has an at least partially adhesive inner surface.

16. The enveloping body as claimed in claim 1 wherein the enveloping body is designed as a prosthesis liner, bandage, cuff, item of clothing or shoe.

17. The enveloping body as claimed in claim 16, wherein the enveloping body, as prosthesis liner, has at least one fastening device for a prosthesis component that is to be secured.

18. The enveloping body as claimed in claim 3, wherein elements transmitting tensile force are arranged between at least two walls.

19. The enveloping body as claimed in claim 18, wherein the elements transmitting tensile force are rigid under tension or elastic.
